(19) United States
(12) Patent Application Publication
(10) Pub. No.: US 2007/0043287 A1
(43) Pub. Date: Feb. 22, 2007

(54) Retrospective triggered mri of active or passive joint motion

(76) Inventor: Frits A. Degraaf, Best (NL)

Correspondence Address: PHILIPS INTELLECTUAL PROPERTY & STANDARDS, 595 MINER ROAD, CLEVELAND, OH, 44143, US

(21) Appl. No.: 10/570,731

(22) PCT Filed: Aug. 30, 2004
(86) PCT No.: PCT/IB04/51611

§ 371 Date: Mar. 6, 2006

Related U.S. Application Data
(60) Provisional application No. 60/501,176, filed on Sep. 8, 2003.

(51) Int. Cl. A61B 5/05

(52) U.S. Cl. 600/410

(57) ABSTRACT

A subject is placed on a patient support (16) with a bodily structure, such as a joint, within an imaging region (12) of a magnetic resonance imager (10). The bodily structure is moved continuously back and forth through a series of motion states, either manually or under power (70, 72). Encoded magnetic resonance data is distributed (42) into motion state windows. Data with phase encodings near the center of k-space are distributed accurately into the corresponding motion state window; whereas, data phase encoded near the edge of k-space is distributed with less accuracy and may be duplicated and shared among several motion state windows. Data from each motion state window are reconstructed (50) and displayed (56) in a cine mode representing continuous motion.

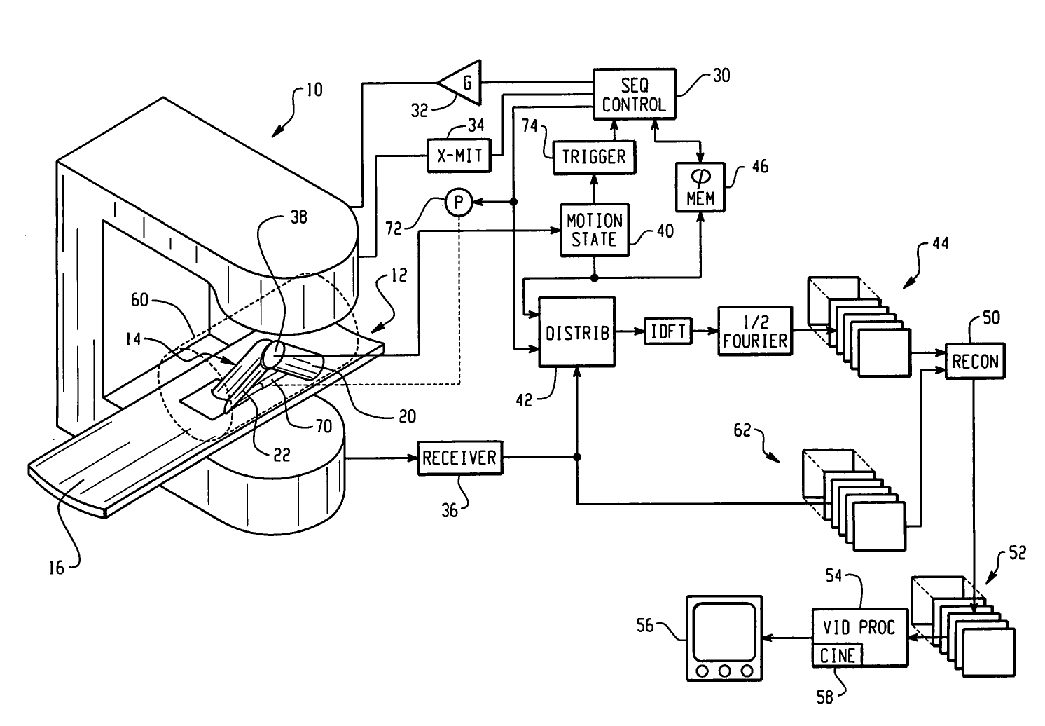

The present technique relates to the magnetic resonance imaging of movable body parts. It finds particular application in conjunction with generating images of a joint in motion, and will be described with particular reference thereto. However, it is to be understood that it is also applicable to other movable body parts, such as the eyes and to autonomously moving body parts as well.

Heretofore, a wide range of cushions and joint positioning devices have been used to hold a subject frozen in a preselected physical position during magnetic resonance imaging. These joint positioning devices were effective to hold the patient against motion during the data acquisition time to eliminate the blurring and other image degradation attributable to patient motion.

When imaging a patient's joints, it is often advantageous to have images of the joint at different degrees of flexation. This was typically done by fixing the joint position and generating a first image. Then, the subject was repositioned with the joint in a different degree of flexation, the position was fixed, and another image was generated. Any number of still images with different degrees of flexation could be generated. U.S. Pat. No. 5,899,859 to Votruba discloses an apparatus for stepping a joint to each of a plurality of predetermined positions and holding the joint still at each position while an MRI image is generated. This series of still images could be displayed sequentially to give the appearance of motion. Although the sequence of images might give the appearance of motion or even continuous motion, they are not images of the joint in motion.

The present application contemplates a new and improved method and apparatus which overcomes the above-referenced problems and others.

In accordance with one aspect of the present invention, a system is disclosed for generating cine images of a continuously moving bodily structure. A magnetic resonance imager generates magnetic resonance image data of a bodily structure of a subject in an examination region as the subject moves through a series of kinematic motion studies. A means provides indications of current motion states through which the bodily structure is moving. A distributing means distributes the magnetic resonance data in accordance with the detected current motion states.

In accordance with another aspect of the present invention, a method is provided for generating cine images of a continuously moving bodily structure. The bodily structure continuously moves back and forth through a series of motion states. Magnetic resonance image data of the bodily structure is generated as it moves continuously through the motion states. Current motion states through which the joint is moving are detected. The magnetic resonance data is distributed in accordance with the detected current motion states.

One advantage of the present invention is that it images bodily structures, such as joints, while in dynamic motion.

Another advantage of the present invention resides in efficiently collecting data for a series of high resolution images.

Other advantages of the present invention include that it can be used for musculo-skeletal studies of a wide range of joints in a variety of magnetic resonance scanning devices.

Still further advantages and benefits of the present invention will become apparent to those of ordinary skill in the art upon reading and understanding the following detailed description of the preferred embodiments.

The invention may take form in various components and arrangements of components, and in various steps and arrangements of steps. The FIGURE is only for purpose of illustrating the preferred embodiments and is not to be construed as limiting the invention.

The FIGURE is a diagrammatic illustration of a magnetic resonance imaging system in accordance with the present invention.

With reference to the FIGURE, a magnetic resonance scanner 10 includes a main magnet for generating a temporally constant B₀ magnetic field through an imaging region 12. The scanner further includes gradient magnetic field coils for generating magnetic field gradients across the imaging region and radio frequency coils for exciting and manipulating resonance in a region of interest of a subject in the examination region and receiving the resultant resonance signals. In one embodiment, a kinematic joint device 14 for moving kinematic studies is mounted to a subject support 16 and disposed in the examination region. The kinematic joint device is strapped or otherwise connected with a patient adjacent a joint to be imaged, positioning the joint preferably at the isocenter of the MR scanner. The kinematic joint device is configured to permit the joint to be flexed, but only along a preselected trajectory. The kinematic joint device can limit the kinematic motion to a plane; can limit motion to rotation along a preselected arc, or the like.

In the illustrated embodiment, the kinematic joint device includes a first portion 20 for fixing a subject's thigh at a preselected, fixed angle to the patient support 16. A second portion 22 which is pivotally connected to the first portion is configured to receive the patient's calf. A pivotal connection between the two portions permits the subject to move the calf up and down along a preselected trajectory, e.g., within a-vertical plane. Of course, various other devices as are known in the art can be used for limiting the motion of knees and other joints.

Alternately, the joint or other movable bodily structure is moved over the selected trajectory-without mechanical assistance. The eyeball, for example, is not well suited for interconnection with a mechanical device, nor are most internal organs.

A sequence controller 30 controls a series of gradient coil amplifiers 32 and a transmitter 34 to initiate a selected imaging sequence, preferably a three-dimensional, high resolution gradient echo sequence. Such a sequence typically generates a line of data in 6-10 milliseconds. The sequence controller causes the imaging sequence to be applied repeatedly as the subject moves the bodily structure repeatedly over the selected trajectory. A receiver 36, which is connected with receive coils either mounted in the scanner or positioned on the joint as localized coils, demodulates resonance signals to generate a data line with each repetition of the imaging sequence. As is typical in the art, data lines which are phase encoded to span k-space are generated.

A motion state indicator means 38, such as an angular position resolver in the kinematic joint device embodiment detects the instantaneous kinematic state of the bodily structure and outputs a signal indicative of the current motion state. Other motion state indicator means are also contemplated such as electro-mechanical devices for sensing linear movement, cameras, laser gauges, MR navigator echoes, and the like. A kinematic motion state circuit 40 converts the output of the motion state indicating means into an appropriate indication of the current kinematic state of the joint as each data line is read. Each data line with its kinematic state information is conveyed to a processor or data distributing means 42 for selecting and distributing the data lines according to motion state and assigning the selected data lines to a plurality of cine image data sets. Any data collected for motion states that are not along the selected trajectory, or are otherwise identified as being inappropriate to the study, are discarded. These k-space data sets are stored in corresponding memory sections 44 of one or a plurality of image data memories. Optionally, a one-dimensional inverse Fourier transform is performed on each data line before it is stored in the data memory 44. More specifically to the illustrated knee joint embodiment, the range of kinematic motion is divided into a plurality of arc segments, preferably equal arc segments. As the patient flexes the joint continuously over the range of motion, kinematic data lines which are generated. The motion state indicating means 38 indicates the current arc segment window through which the subject is moving and the current window and the data lines are stored in a corresponding data window subset of the data memory 44. Preferably, the arc segments for the centrally phase-encoded views are relatively small, with the arc segments for the peripheral phase-encoded views being larger. The subject flexes the joint back and forth over the motion range. As may be appropriate to the study, data lines are generated either during both back and forth motion or only during motion in one direction.

Various distributing techniques are contemplated. In one embodiment, the arc segment windows are defined based on physical motion segments along the trajectory and the data acquisition continues until a full data set is collected in the window for each motion state. Optionally, a data line memory 46 keeps a record of the phase encode steps for which data lines have been collected for each motion window. The sequence controller 30 adjusts the phase encoding order to generate data lines with needed phase encoding in the various motion segments. As another option, the data distributing means makes a “best fit” division of the data lines among the windows, with the fit parameters being stricter for centrally phase encoded data lines and progressively looser for higher phase encodings. Alternately, the higher phase encoded data lines can be distributed into two or more adjacent motion state windows. Further, data lines can be replaced by newly acquired data to improve the fit with which the data lines are matched to the motion states. Numerous other distributing techniques are also contemplated. For example, rather than fixing the range of motion corresponding to each window, the minimum amount of data per window can be specified. When data in excess of the minimum is collected, the window can be divided into two or more new windows corresponding to smaller ranges of motion and the data redistributed.

Once the set of k-space data lines for each of the motion segments has been collected, preferably three-dimensional data sets, a reconstruction processor 50 reconstructs each data set into a corresponding image, preferably a 3D image, which is stored in sections of an image memory 52. A video processor 54 withdraws selected portions of the 3D images for display on a video monitor 56. Preferably, the video processor includes a cine processor 58 which withdraws corresponding images sequentially from each of the motion state images to produce a moving or cine display of the joint motion. The displayed cine images may be slice images, 3D renderings, or other image representations as are known in the art. The various tissues can be displayed with different coloration, with some tissue removed or invisible, e.g., with the bones transparent to view the cartilage, muscle, patella, meniscal fluid, other soft tissue directly, and the like. Reference information such as central axes of bones, highlighted surface contours, radius of curvature, dimensions, and the like can be superimposed on the images.

In a real time acquisition mode, the central data lines are collected first with data collection moving from the center of k-space progressively outward. Optionally, peripheral portions of k-space are zero-filled until actual data is collected. The reconstruction processor continuously or semi-continuously updates the reconstructed images as more data is collected. The displayed images improve in resolution and detail in real time as more data is collected. The operator can terminate the scan when images of a satisfactory quality for the current diagnostic purposes have been generated.

Optionally, a half-Fourier processor uses conjugate symmetry to create synthetic data lines on the opposite side of the centrally encoded data lines for each actually collected data line.

As another option, a receive coil 60 that has multiple antennae for parallel imaging, such as a SENSE coil, can be used to collect data in fewer repetitions. The SENSE coil can be attached to or adjacent the region of interest to move with it or can be stationarily mounted to the scanner. To compensate for changes in sensitivity characteristics of the antennae with motion state, low resolution, central k-space reference data are generated for each motion state and stored in reference data memories 62. The reference data sets are used to generate an inversion matrix for each motion state window which are used to unfold aliased parallel images into unaliased images. In a preferred embodiment, the continuous motion data is undersampled, and the reference data is used as a regularization image to improve the conditioning of the matrix inversion. Optionally, the reference and continuous motion data can be generated with different contrasts to create related images with different contrasts for emphasizing a selected tissue.

As an additional option, multiple scans with different MR contrasts (e.g., T₁, T₂, steady-state free precession, water-only) are acquired to improve the diagnostic value of the examination. In that case, it is preferred to acquire the reference data for all relevant motion states only once, in a separate, specifically tailored scan.

Rather than the patient flexing the joint freely through the motion states, a pneumatic cylinder 70 or other resistance means is optionally provided to provide a selectable force against which the patient must work to flex the joint.

In yet another alternate embodiment, the sequence control circuit 30 is connected with a controller 72, such as pneumatic controller, which controls the pneumatic cylinder 72 or other motor means to control the movement of the kinematic motion device. In this manner, the sequence control means can control the motion state dynamically to move the joint continuously through the range of motion. Because the sequence controller is both controlling the continuous motion and the phase encoding, it optimizes the efficiency with which the phase encoding data for each of the motion range windows is generated. By knowing the position which the joint will be in before the phase encoding is applied, the sequence controller can select only phase encoding steps which are needed for the window that the joint will be in when the data line is generated and eliminate the collection of redundant or superfluous data. Further, more centrally phase encoded data lines can be generated nearest the centers of the windows.

In yet another alternate embodiment, the motion state indicator circuit 40 is connected with a triggering means 74 which triggers or steps the sequence controller 30 to apply the appropriate phase encoding to collect k-space data lines that are needed in the kinematic motion state window in the current state of motion. The triggering means 74 and the sequence controller 30 interact with the view memory 46 to determine the phase encode steps which are still needed for each motion state window as it is entered.

The invention has been described with reference to the preferred embodiments. Modifications and alterations will occur to others upon a reading and understanding of the preceding detailed description. It is intended that the invention be construed as including all such modifications and alterations insofar as they come within the scope of the appended claims or the equivalents thereof.

Having thus described the preferred embodiments, the invention is now claimed to be:

1. A system for generating cine images of a continuously moving bodily structure, the apparatus comprising:
a magnetic resonance imager (10) which generates magnetic resonance image data of a bodily structure of a subject in an examination region (12) as the subject bodily structure moves continuously through a continuous series of motion states;
a means (38, 40, 72) for providing indications of current motion states through which the bodily structure is moving; and,
a distributing means (42) for distributing the magnetic resonance data in accordance with the detected current motion states.

2. The apparatus according to claim 1, wherein the distributing means (42) distributes the magnetic resonance data among a plurality of motion state windows and further including:
a sequence controller (30) which cause the magnetic resonance imager (10) to generate phase encoded data with each of a plurality of preselected phase encoding steps within each of the motion state windows as the bodily structure moves continuously back and forth through the motion states.

3. The apparatus according to claim 2, further including:
a reconstruction means (50) for reconstructing the magnetic resonance data corresponding to each motion state window into an image representation;
a cine means (58) for presenting the image representations in a cine mode such that images displayed on a monitor (56) depict continuous movement of the bodily structure.

4. The apparatus according to claim 3, further including:
a means (74) for signaling to the sequence controller each time the bodily structure moves into a next motion state window.

5. The apparatus according to claim 3, further including:
a memory (46) for providing an indication to the sequence controller of the phase encode steps for which data has already been collected corresponding to each motion state window.

6. The apparatus according to claim 3, wherein the distributing means (42) distributes the data with a near central phase encoding into a single motion state window and data with high phase encodings into a plurality of motion state windows.

7. The apparatus according to claim 3 wherein the distributing means (42) distributes the data with near central phase encoding into narrower motion state windows than data with higher phase encoding.

8. The apparatus according to claim 1, wherein the bodily structure is a joint and further including:
a kinematic joint device (14) which constrains motion of the joint to a selected trajectory.

9. The apparatus according to claim 8, further including:
a means (70, 72) for moving the kinetic joint device (14) continuously along the trajectory.

10. The apparatus according to claim 9, wherein the sequence controller (30) controls the means (70, 72) for moving the kinematic point device (14).

11. A method for generating cine images of a continuously moving bodily structure, the method comprising:
continuously moving the bodily structure back and forth through a series of motion states;
generating magnetic resonance image data of the bodily structure as it moves continuously through the motion states;
detecting current motion states through which the bodily structure is moving; and,
distributing the magnetic resonance data in accordance with the detected current motion states.

12. The method according to claim 11 wherein the data is distributed among a plurality of motion state windows, and further including:
generating phase encoded data with each of a plurality of preselected phase encodings within each of the motion state windows.

13. The method according to claim 12, further including:
reconstructing the magnetic resonance data corresponding to each motion state window into an image representation;
displaying the image representations in a cine mode such that the image display depicts continuous movement of the bodily structure.

14. The method according to claim 12, wherein determining the current motion state includes one of:
generating navigator echoes;
optically imaging the continuously moving bodily structure; and,
monitoring a position a portion of a kinematic device connected with the bodily structure.

15. The method according to claim 12, further including:
updating a record of the phase encoding with which data has been collected corresponding to each motion state window.

16. The method according to claim 13, further including:
generating low resolution reference data for each motion state window with a plurality of antennae;
using the reference data as a regularization image to improve conditioning of an inversion matrix for each of a plurality of the motion states for unfolding aliased images collected in parallel from the plurality of antennae during the continuous motion through the motion states;
using the reference data in conjunction with the image data from the continuously moving bodily structure to reconstruct the cine mode image representations.

17. The method according to claim 13, further including:
generating the image data from a center of k-space progressively outward;
repeatedly reconstructing the image data such that the cine mode image display improves as more data is collected;
stopping the generating of image data after the cine mode image display becomes satisfactory.

18. The method according to claim 12, wherein the data is distributed at least one of:
with near central phase encodings into a single motion state window and data with high phase encodings duplicated and shared among a plurality of phase encode windows;
with data with nearer central phase encoding sorted among narrower motion state windows than data with higher phase encoding; and
into progressively narrower motion state windows as additional image data is generated.

19. The method according to claim 11 wherein the bodily structure is a joint and further including:
mechanically constraining the joint to move back and forth along a selected trajectory.

20. The method according to claim 19 wherein each motion state window corresponds to an equal portion of the trajectory.

21. A computer program for programming a magnetic resonance imager (10) to generate cine images of a bodily structure which is continuously moving back and forth through a series of motion states by performing the steps of:
generating magnetic resonance image data of the bodily structure as it moves continuously through the motion states;
detecting current motion states through which the bodily structure is moving; and,
distributing the magnetic resonance data in accordance with the detected current motion states.
